(19) United States
(12) Patent Application Publication
(10) Pub. No.: US 2023/0233510 A1
(43) Pub. Date: Jul. 27, 2023

(54) CYSTEINE PROTEASE INHIBITORS FOR USE IN THE PREVENTION AND/OR TREATMENT OF CORONAVIRUS

(71) Applicant: ALBERT-LUDWIGS-UNIVERSITÄT FREIBURG, FREIBURG (DE)

(72) Inventors: CHRISTOPH PETERS, BAHLINGEN A.K. (DE); SEBASTIAN FÄHNDRICH, MALTERDINGEN (DE); MARTIN HUG, FREIBURG (DE)

(21) Appl. No.: 17/919,048

(22) PCT Filed: Apr. 16, 2021
(86) PCT No.: PCT/EP2021/059964

§ 371 Date: Oct. 14, 2022

(30) Foreign Application Priority Data
Apr. 16, 2020 (DE) 10 2020 110 449.9

(51) Int. Cl. A61K 31/336; A61P 31/14; A61K 31/706; A61K 38/19; A61K 38/55

(52) U.S. Cl. CPC A61K 31/336; A61P 31/14; A61K 31/706; A61K 38/19; A61K 38/55

(57) ABSTRACT

The present invention relates to a compound according to formula (I) Formula (I) a physiologically acceptable salt, a solvate, or a hydrate thereof for use in the prevention and/or treatment of diseases caused by betacorona-virus infection in a mammalian subject, such as a human, wherein said compound is administered by inhalation as a pharmaceutical composition comprising preferably alcohol as a carrier. A preferred example is viral infection caused by SARS.

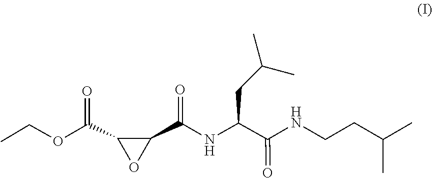 [SMILES: CCOC(=O)[C@H]1O[C@@H]1C(=O)N[C@@H](CC(C)C)C(=O)NCCC(C)C]

The present invention relates to a compound according to formula (I) or a salt, solvate and/or a hydrate thereof, wherein said compound inhibits a cysteine protease, for use in the treatment and prevention of viral infection with coronavirus, wherein said compound is administered by inhalation as a pharmaceutical composition, preferably comprising alcohol as a carrier. The present invention particularly relates to viral infections caused by betacoronavirus, in particular by SARS-CoV-2.

### BACKGROUND OF THE INVENTION

Betacoronaviruses (β-CoVs or Beta-CoVs) are one of four genera of coronaviruses of the subfamily Orthocoronavirinae in the family Coronaviridae, of the order Nidovirales. They are enveloped, positive-sense, single-stranded RNA viruses of zoonotic origin.

Recently, a novel coronavirus emerged in the Chinese city of Wuhan in December 2019. After human coronavirus 229E (HCoV-229E) (classified in the genus Alphacoronavirus) and HCoV-OC43 (Betacoronavirus lineage 2a member) described in the 1960s, SARS-CoV-1 (Betacoronavirus lineage 2b member) that emerged in March 2003, HCoV-NL63 (Alphacoronavirus lineage 1b member) described in 2004, HCoV-HKU1 (Betacoronavirus lineage 2a member) discovered in 2005, and finally MERS-CoV that emerged in 2012 (classified in Betacoronavirus lineage 2c), the novel coronavirus is the seventh human coronavirus described to date as being responsible for respiratory infection. Evidence emerged that patients were suffering from an infection with a novel Betacoronavirus tentatively named 2019 novel coronavirus (2019-nCoV). Despite drastic containment measures, the spread of 2019-nCoV, now officially known as severe acute respiratory syndrome coronavirus 2 (SARS-CoV-2), is ongoing. Phylogenetic analysis of this virus indicated that it is different (˜80% nucleotide identity) but related to SARS-CoV-1.

It appears that there are two distinct but overlapping pathological subsets of COVID-19, the first triggered by the virus itself and the second, the host response. The initial stage occurs at the time of inoculation and early establishment of disease. For most people, this involves an incubation period associated with mild and often non-specific symptoms such as malaise, fever and a dry cough. At this stage, it is reported that the virus replicates in the upper respiratory tract.

Treatment at this stage is primarily aimed towards symptomatic relief. In the second stage of established pulmonary disease, viral multiplication and localized inflammation in the lung is the norm. During this stage, patients develop a viral pneumonia, with cough, fever and possibly hypoxia. Treatment would primarily consist of supportive measures and available antiviral therapies. If hypoxia ensues (Stage IIb), it is likely that patients will require mechanical ventilation while the use of anti-inflammatory therapy may be useful. A minority of COVID-19 patients will transition into the third and most severe stage of illness, which manifests as an extra-pulmonary systemic hyperinflammation syndrome. In this stage, markers of systemic inflammation appear to be elevated, including cytokines and interleukins. In this stage, shock, vasoplegia, respiratory failure and even cardiopulmonary collapse are discernable. Systemic organ involvement, even myocarditis, may manifest during this stage. Tailored therapy in stage III hinges on the use of immunomodulatory agents to reduce systemic inflammation before it overwhelmingly results in multi-organ dysfunction.

As of April 2020, no vaccine was available and, although numerous pharmacological interventions are under clinical investigation, clear efficacy has not yet been determined for any drug.

The protease inhibitor aloxistatin (alternative names: E 64 D; EST; Estate; Loxistatin; Rexostatine) is a synthetic analogue of E 64, a natural product derived from fungi. E 64 D is the ethyl ester of E 64 c, and seems to hydrolyzed back to E 64 c as the more active form during the permeation through intestinal membrane (i.e. after oral uptake). The formula of aloxistatin is as follows:

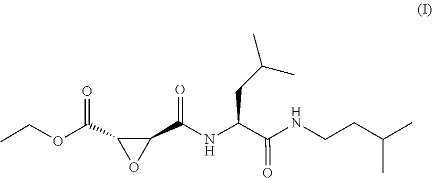 [SMILES: CCOC(=O)[C@H]1O[C@@H]1C(=O)N[C@@H](CC(C)C)C(=O)NCCC(C)C]

Chemically, E 64 D is an L-leucine derivative that is the amide obtained by formal condensation of the carboxy group of (2S,3S)-3-(ethoxycarbonyl)oxirane-2-carboxylic acid with the amino group of N-(3-methylbutyl)-L-leucinamide. It is thus an L-leucine derivative, a monocarboxylic acid amide, an epoxide and an ethyl ester. E 64 D is an inhibitor of cysteine proteases, cathepsins B and L, and is also thought to inhibit calpain.

E 64 D was initially developed for the treatment of muscular dystrophy but was not successful in human clinical trials, though it has continued to be investigated for treatment of spinal cord injury, stroke and Alzheimer's disease. E 64 D has also been described to have some antiviral effects.

Kim et al. (in: Coronavirus protein processing and RNA synthesis is inhibited by the cysteine proteinase inhibitor E64d. Virology. 1995 Apr. 1; 208(1):1-8) that E64D inhibits replication of MHV-A59 in murine DBT cells in a dose-dependent manner, resulting in reduced virus titers and viral syncytia formation. SARS-CoV and HCoV-NL63 would use angiotensin-converting enzyme 2 (ACE2) as their receptor, MHV enters through CEACAM1, and the recently identified MERS-CoV would bind to dipeptidyl-peptidase 4 (DPP4) to gain entry into human cells. The genomic organization of MHV is quite different from, e.g., SARS-CoV (see Fehr, Anthony R.; Perlman, Stanley (2015). “Coronaviruses: An Overview of Their Replication and Pathogenesis”. Coronaviruses. Methods in Molecular Biology 1282. pp. 1-23).

Van der Linden W A et al. (in: Cysteine Cathepsin Inhibitors as Anti-Ebola Agents. ACS Infect Dis. 2016 Mar. 11; 2(3):173-179. Epub 2016 Jan. 20) disclose E-64 as an ideal starting point for the development of antifilovirus drugs targeting these enzymes. Nevertheless, the poor membrane permeability of E-64 and analogues would limit their antiviral potency, as they must be added to cells at high concentrations to sufficiently permeate lysosomes and block CatB and CatL activities. Esterification of the free carboxylic acid in E-64 alleviates this problem in tissue culture, but these esters are not suitable for in vivo administration, because they are rapidly and efficiently hydrolyzed by esterases abundant in plasma. In order to generate a small focused library of inhibitors, they used the main core structure of E64 (AMS36) but coupled a set of 24 drug-like amines to the Phe(Me)-epoxysuccinate, and subsequent screening yielded highly potent, broad-spectrum inhibitors of the lysosomal cysteine cathepsins.

Kawase M et al. (in: Simultaneous treatment of human bronchial epithelial cells with serine and cysteine protease inhibitors prevents severe acute respiratory syndrome coronavirus entry. J Virol. 2012 June; 86(12):6537-45. doi: 10.1128/JVI.00094-12. Epub 2012 Apr. 11) disclose that the type II transmembrane protease TMPRSS2 activates the spike (S) protein of severe acute respiratory syndrome coronavirus (SARS-CoV) on the cell surface following receptor binding during viral entry into cells. In the absence of TMPRSS2, SARS-CoV achieves cell entry via an endosomal pathway in which cathepsin L may play an important role, i.e., the activation of spike protein fusogenicity. A commercial serine protease inhibitor (camostat) partially blocked infection by SARS-CoV and human coronavirus NL63 (HCoV-NL63) in HeLa cells expressing the receptor angiotensin-converting enzyme 2 (ACE2) and TMPRSS2. Simultaneous treatment of the cells with camostat and EST [(23,25)trans-epoxysuccinyl-L-leucylamindo-3-methylbutane ethyl ester], a cathepsin inhibitor, efficiently prevented both cell entry and the multistep growth of SARS-CoV in human Calu-3 airway epithelial cells. This efficient inhibition could be attributed to the dual blockade of entry from the cell surface and through the endosomal pathway. These observations suggest camostat as a candidate antiviral drug to prevent or depress TMPRSS2-dependent infection by SARS-CoV.

Similarly, Hoffmann M et al. (in: SARS-CoV-2 Cell Entry Depends on ACE2 and TMPRSS2 and Is Blocked by a Clinically Proven Protease Inhibitor. Cell. 2020 Mar. 4. pii: S0092-8674(20)30229-4. doi: 10.1016/j.cell.2020.02.052) disclose that SARS-CoV-2 uses the SARS-CoV receptor ACE2 for entry and the serine protease TMPRSS2 for S protein priming. A TMPRSS2 inhibitor approved for clinical use blocked entry and might constitute a treatment option. SARS-CoV is able to use the endosomal cysteine proteases cathepsin B and L (CatB/L) and the serine protease TMPRSS2, but only TMPRSS2 activity is essential for viral spread and pathogenesis in the infected host, whereas CatB/L activity is dispensable. Therefore, a TMPRSS2 inhibitor approved for clinical use is proposed to block viral entry as constituting a treatment option. The publication actually teaches away from the use of E 64 D as “only” blocking entry through cathepsin B and L, which can be circumvented by TMPRSS2.

US 2004/0235952 discloses inhibitors of severe acute respiratory syndrome (SARS) 3C-like proteinases. The application relates to methods of inhibiting SARS-related coronavirus viral replication activity comprising contacting a SARS-related coronavirus protease with a therapeutically effective amount of a rhinovirus protease inhibitor, and compositions comprising the same. The inhibitor can be administered orally, intravenously or by inhalation. E64D seems not disclosed.

US 2019/0151400 discloses protease transition state inhibitor/analogue prodrug compounds selected from the group consisting of esters, carbamates, ester phosphates, and pharmaceutically acceptable salts thereof. Compositions containing such prodrugs are also disclosed, along with methods of using such compounds therapeutically or prophylactically against calicivirus, picornavirus, and/or coronavirus infection. The prodrug compound inhibits 3C or 3C-like protease activity of one or more viruses. It can further comprising polyethylene glycol covalently attached to said compound.

It is an object of the present invention to provide effective agents that can be used for the prevention and treatment of viral infection that can be treated/prevented by the inhibition of cysteine proteases, in particular coronavirus infection. Other objects and advantages will become apparent to the person of skill when studying the present description of the present invention.

In a first aspect of the present invention, this object is solved by a compound according to Formula (I)

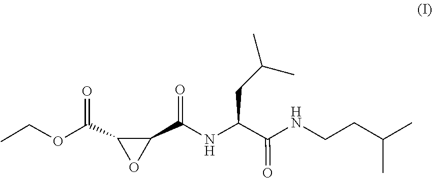 [SMILES: CCOC(=O)[C@H]1O[C@@H]1C(=O)N[C@@H](CC(C)C)C(=O)NCCC(C)C]

(aloxistatin, E 64 D; EST; Estate; Loxistatin; Rexostatine), a physiologically acceptable salt, a solvate, or a hydrate thereof for use in the prevention and/or treatment of diseases caused by betacoronavirus infection in a mammalian subject, such as a human, wherein said compound is administered by inhalation as a pharmaceutical composition preferably comprising alcohol as a carrier.

It was surprisingly found that the compound according to Formula I (aloxistatin, E 64 D; EST; Estate; Loxistatin; Rexostatine), as a cysteine protease inhibitor, was effective and demonstrates many advantages compared to other antiviral treatment options. E 64 D shows low toxic effects as an inhibitor, in addition to its effective mechanism of action. It blocks both viral entry and replication, is safe, and can be conveniently provided as an oral dosage and/or inhaled. According to a preferred aspect of the invention, E 64 D is inhaled in order to prevent progression of the viral infection towards a more severe stage, in particular to prevent the need for mechanical ventilation, a major problem in case of large numbers of infected patients.

When orally administering aloxistatin (E64D), the compound is cleaved by esterases that are present in the blood into its acid form, loxistatin (E64C), which is markedly less effective in blocking viral entry into host cells and may not reach the luminal surface of the airways altogether. Since only the ester compound aloxistatin can pass the cellular membrane as a lipophilic pro-drug, and furthermore blocks intracellular cathepsins that are also involved in the introduction of viruses into the host cell, a higher efficiency of the drug is ensured when using inhalation. In addition, the topical application using inhalation leads to higher local doses than with oral gavage.

In the context of the present invention, the term “aloxistatin” or “E 64 D” shall include the compound according to formula I in its physiologically acceptable salt forms, such as the calcium, potassium, magnesium, choline or sodium salt. The term shall also include physiologically acceptable solvates, and hydrates. As a prodrug, it shall also include E 64 c, which seems to be the more active form following the permeation through intestinal membrane (i.e. after oral uptake). The formula of E 64 c is as follows:

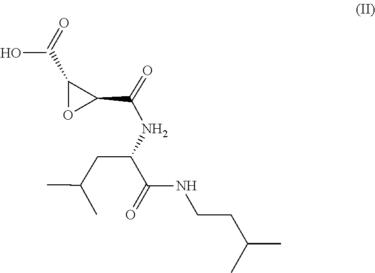

Preferred is the compound for use according to the present invention, wherein said viral infection is by a virus selected from the viral groups represented by HCoV-OC43, SARS-CoV-1, HCoV-HKU1, MERS-CoV or SARS-CoV-2 and/or the G614 variant thereof, which are particularly preferred.

Antiviral effects by protease inhibition have been described for K11777, camostat, or similar serine protease inhibitors, or other compounds as described in the references above.

Certain features of the coronavirus spike (S) protein optimize the virus towards different parts of the respiratory tract. The SARS-CoV-2 spike protein reaches higher levels in pseudoparticles when produced at 33° C. instead of 37° C. Furthermore, the D614G mutation in SARS-2-S increases S protein stability and expression at 37° C., and promotes virus entry via cathepsin B/L activation. These spike properties might promote virus spread, potentially explaining why the G614 variant is currently predominating worldwide. This shows how the coronavirus spike protein is fine-tuned towards the temperature and protease conditions of the airways, to enhance virus transmission and pathology (Laporte, M. et al. The SARS-CoV-2 and other human coronavirus spike proteins are fine-tuned towards temperature and proteases of the human airways. bioRxiv 2020.11.09.374603; doi: haps://doi.org/10.1101/2020.11.09.374603).

It has even been shown that E64D is particularly effective in the early phases of the disease, e.g. 12-16 hours after infection, while other antiviral drugs are able to inhibit viral infection up to 4 hours after infection, E64D is particularly effective in the early phases of the disease, e.g. 12-16 hours after infection. Furthermore, a broad spectrum effect both on viral and host proteases can be assumed with E64D. The papain-like protease encoded in SARS-CoV-2, PL-Pro, has a structure very similar to that of cathepsines B and L that is irreversible inhibited by E-64D and therefore constitutes a preferred protease for the inhibiting effect of the compound(s) for use.

According to the present invention, a mammalian subject or patient can be preferably selected from a mouse, rat, cat, dog, rabbit, goat, sheep, horse, camel, lama, cow, monkey, a farm animal, a sport animal, and a pet, and a human, in particular an infected child (i.e. 0-9 years of age).

According to the present invention, the compound is for use in viral infection, and therefore treats and/or prevents the related diseases or syndromes, such as, for example, respiratory disease, acute respiratory disease, sepsis, acute respiratory distress syndrome, and adverse immune reactions, in particular moderate to severe cases of said diseases, and wherein said disease preferably is selected from diseases causes by SARS-CoV-2, in particular COVID-19, such as acute respiratory disease, sepsis, acute respiratory distress syndrome, and adverse immune reactions, such as a cytokine storm. It is expected that the pharmaceutical composition comprising the present compound for use is particularly effective in infected children (i.e. 0-9 years of age).

It is expected that the present treatment is particularly effective in children (0-9 years of age), since non-mutant SARS-CoV2-viruses require TMPRSS2 in order to infect host cells. Children express low amounts of TMPRSS2 and therefore were not affected as much by CoViD-19. Nevertheless, novel mutants infect cells independently from TMPRSS2, and it is hypothesized that children can develop severe infections, because alternative pathways, such as cathepsines, allow for viral entry. Therefore, inhalation provides an effective tool to treat children that are infected with mutants of the “classical” SARS-CoV2-Virus, such as the G614 variant.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms. By “amelioration” is meant the prevention, reduction or palliation of a state, or improvement of the state of a subject; the amelioration of a stress is the counteracting of the negative aspects of a stress. Amelioration includes, but does not require complete recovery or complete prevention of a stress.

In another important aspect according to the present invention regarding the compound for use, said prevention and/or treatment is in combination with another antiviral therapy, for example selected from at least one remdesivir, interferon alpha 2a or 2b, inclusive any pegylated versions, chloroquine or hydroxychloroquine, serine protease inhibitors, cysteine protease inhibitors, and/or type II transmembrane protease (TMPRSS2) inhibitors, in particular camostat ((4-{2-[2-(Dimethylamino) oxoethoxy]-2-oxoethyl}phenyl)(4-carbamimidamidobenzoate), inhalable corticosteroids, vasointestinal peptide (VIP) and furin inhibitors. Preferred is a combination with remdesivir, chloroquine or hydroxychloroquine, and/or camostat, which shows a synergistic effect. Kawase, M., et al. (in: Simultaneous Treatment of Human Bronchial Epithelial Cells with Serine and Cysteine Protease Inhibitors Prevents Severe Acute Respiratory Syndrome Coronavirus Entry. Journal of Virology May 2012, 86 (12) 6537-6545; DOI: 10.1128/JVI.00094-12) disclose that a simultaneous treatment of the cells with camostat and EST [(23,25)trans-epoxysuccinyl-1-leucylamindo-3-methylbutane ethyl ester], a cathepsin inhibitor, efficiently prevented both cell entry and the multistep growth of SARS-CoV in human Calu-3 airway epithelial cells. The present invention can be adjusted accordingly, i.e. be combined with a TMPRSS2 inhibitor.

The compound for use is provided and/or is administered as a suitable pharmaceutical composition for inhalation, such as a dry powder inhaler or other inhalation forms. Such formulations may comprise excipients and other ingredients in suitable amounts.

In the context of the present invention, an efficient inhalation furthermore requires the presence of a suitable solvent. The choice is not trivial, as for an effective inhalation it is required that the drug does not precipitate, and stays in solution. Due to the lipophilic nature of the compound only organic solvents can be used for a liquid formulation. Preferably, alcohol is used as a suitable solvent, the inventors found that ethanol, which itself has a disinfecting and antiviral effect, proved to be a suitable with a solubility of E64D of up to 68 mg/mL (198.58 mM). In addition, ethanol can be obtained in purified/GMP grade quality and is approved as additive for use in humans. The person of skill is able to identify respective concentrations that are suitable for the purposes of the present application.

It is to be understood that the present compound and/or a pharmaceutical composition comprising the present compound is for use to be administered to a human patient, and it is expected that the pharmaceutical composition comprising the present compound for use is particularly effective in infected children (i.e. 0-9 years of age). The term “administering” means administration of a sole therapeutic agent or in combination with another therapeutic agent. It is thus envisaged that the pharmaceutical compositions of the present invention are employed in co-therapy approaches, i.e. in co-administration with other medicaments or drugs and/or any other therapeutic agent which might be beneficial in the context of the methods of the present invention. Nevertheless, the other medicaments or drugs and/or any other therapeutic agent can be administered separately from the compound for use, if required, as long as they act in combination (i.e. directly and/or indirectly, preferably synergistically) with the present compound for use.

Thus, the compounds for use of the invention can be used alone or in combination with other active compounds—for example with the aforementioned compounds, whereby in the latter case a favorable additive, amplifying or preferably synergistically effect is noticed. Suitable amounts to be administered to humans range from 1 to 500 mg, in particular 5 mg to 100 mg, such as between 1 and 10 mg/kg/day E64D dose. An effective concentration to be reached at the cellular level can be set at between 50 to 200 μM, preferably at about 100 μM. For topical application, such as to the airways by inhalation, the dosage can be conveniently reduced to between 0.1 to 10 mg/dose, preferably 0.2 to 5 mg per dose, which equals about 3 to about 80 μg per kilogram for a 70 kg subject.

In the context of the present invention, an efficient inhalation furthermore requires the choice of a suitable volume to be nebulized and inhaled, and thus the resulting drug concentration, on the one hand providing a sufficient dose in all sections of the respiratory tract (nasopharynx, upper, middle respiratory airways and alveolar surface) in order to ensure an inhibition of the entry of SARS-CoV-2 into the host cells. On the other hand, the medical limits for the inhalation of ethanol must be taken into account, while precipitation must be avoided. The person of skill is able to identify respective volumes and concentrations that are suitable for the purposes of the present invention. Also patient compliance is essential, in particular in view of difficulties with breathing in case of COVID-19.

Another important aspect of the present invention is the choice of the technique for inhalation. A preferred technology should avoid a loss of drug during inhalation and at the same time ensure a sufficient dose in all targeted sections of the respiratory system. For the present invention, administration using a breath-triggered nebulizer, for example an M-neb® dose+ nebulizer (Nebutec, Elsenfeld, Germany).

Pharmaceutical compositions as used may optionally comprise a pharmaceutically acceptable carrier. Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol, alcohol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for E64D and will readily be able to choose suitable pharmaceutically acceptable carriers or excipients, depending, e.g., on the formulation and administration route of the pharmaceutical composition.

Preferred is the compound for use according to the present invention, wherein said suitable solvent is an alcohol, such as ethanol.

Yet another important aspect of the present invention is the choice of a suitable particle size that is aerodynamically suitable to reach all of the targeted sections of the respiratory system (see above), while at the same time ensuring satisfying dosage deposition. The person of skill is able to identify respective sizes and particles that are suitable for the purposes of the present invention (see, for particles, for example, El-Sherbiny, Ibrahim M et al. “Inhaled nano- and microparticles for drug delivery.” Global cardiology science & practice vol. 2015 2. 31 Mar. 2015, doi:10.5339/gcsp.2015.2). Suitable average particle diameter sizes are between about 2 and about 7 μm, preferably between about 3 and about 5 μm.

In the context of the present invention, the term “about” shall mean +/−10% of a given value, unless noted otherwise.

The combination therapeutics can be administered orally, e.g. in the form of pills, tablets, coated tablets, sugar coated tablets, hard and soft gelatin capsules, solutions, syrups, emulsions or suspensions or as aerosol mixtures. Administration, however, can also be carried out rectally, e.g. in the form of suppositories, or parenterally, e.g. in the form of injections or infusions, or percutaneously, e.g. in the form of ointments, creams or tinctures. Preferred is also administration using a dry powder inhaler or other form of inhalation.

In addition to the aforementioned compounds for use of the invention, the pharmaceutical composition can contain further customary, usually inert carrier materials or excipients. Thus, the pharmaceutical preparations can also contain additives, such as, for example, fillers, extenders, disintegrants, binders, glidants, wetting agents, stabilizers, emulsifiers, preservatives, sweetening agents, colorants, flavorings or aromatizers, buffer substances, and furthermore solvents or solubilizers or agents for achieving a depot effect, as well as salts for changing the osmotic pressure, coating agents or antioxidants. They can also contain the aforementioned salts of two or more compounds for use of the invention and also other therapeutically active substances as described above.

Preferred is the compound for use according to the present invention, wherein said compound is administered to said subject in an effective dosage. This dosage can vary within wide limits and is to be suited to the individual conditions in each individual case. For the aforementioned uses the appropriate dosage will vary depending on the mode of administration (here, inhalation), the particular condition to be treated and the effect desired. In general, however, satisfactory results are achieved at dosage rates are as above, e.g. of about 1 to 100 mg/kg animal body weight particularly 1 to 50 mg/kg. Suitable dosage rates for larger mammals, for example humans, are of the order of from about 5 mg to 3 g/day, conveniently administered once or in divided doses, e.g. 2 to 4 times a day, preferably 3 time as day, or in sustained release form. In general, a daily dose of approximately 10 mg to 100 mg, particularly 10 to 50 mg, per human individual is appropriate in the case of the oral administration. An effective concentration to be reached at the cellular level can be set at between 50 to 200 μM, preferably at about 100 μM. Particularly preferred is topical application, such as to the airways by inhalation. In these cases, the dosage can be conveniently reduced to between 0.1 to 10 mg/dose, preferably 0.2 to 5 mg per dose, which equals about 3 to about 80 μg per kilogram for a 70 kg subject.

In the context of the present invention, it was surprisingly found that the compound E64D for use according to the present invention shows particular advantages both in the prevention of viral infection as well as in the treatment of later stages of the diseases, particularly as a combination treatment. Particularly effective is a treatment of the G614 variant of SARS-CoV-2.

In the preventive approach, in order to maximize its impact on viral replication and the chance of a successful outcome, it is desired to initiate patient treatment with E64D as soon as possible following the diagnosis of infection. Nevertheless, the compound for use according to the present invention can be administered to a subject later and is still effective, e.g. when administered at between 8 to 24 hours post infection, preferably at between 10 to 20 hours post infection, more preferably at 12 to 16 hours post infection. This regimen still provides a surprisingly low viral load.

In the treatment approach, the compound for use according to the present invention can be administered to a subject “late” in the infection cycle, and is still effective, in particular as a (synergistic) combination therapy, e.g. when administered at between 5 to 14 days post infection, preferably at between 5 to 11 days post infection, more preferably at 7 to 11 days post infection. This regimen provides a surprisingly effective treatment effect, and furthermore helps to control excessive immune reactions, like cytokine storms.

In another aspect thereof, the present invention provides methods for preventing and/or treating of diseases caused by betacoronavirus infection in a mammalian subject, such as a human, comprising administering to said mammal an effective amount of a compound according to Formula (I),

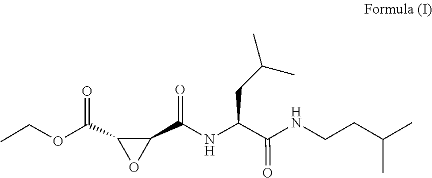 [SMILES: CCOC(=O)[C@H]1O[C@@H]1C(=O)N[C@@H](CC(C)C)C(=O)NCCC(C)C]

a physiologically acceptable salt, a solvate, or a hydrate thereof, wherein said compound is administered by inhalation as a pharmaceutical composition, preferably comprising alcohol as a carrier. Preferably, said viral infection is by viruses of a family exemplified by HCoV-OC43, SARS-CoV-1, HCoV-HKU1, MERS-CoV or SARS-CoV-2, such as the G614 variant of SARS-CoV-2.

The method comprises treating and/or ameliorating symptoms associated with betacoronavirus infection in a mammalian subject, comprising administering to the subject E64D in a pharmaceutically effective amount, and by said administering, reducing symptoms associated with said viral infection, such as symptoms of respiratory disease, acute respiratory disease, sepsis, acute respiratory distress syndrome, and adverse immune reactions, in particular moderate to severe cases of said diseases, and wherein said disease preferably is selected from diseases causes by SARS-CoV-2, in particular COVID-19, such as acute respiratory disease, sepsis, acute respiratory distress syndrome, and adverse immune reactions, such as a cytokine storm.

Preferred is the method according to the present invention, wherein said viral infection is by HCoV-OC43, SARS-CoV-1, HCoV-HKU1, MERS-CoV or SARS-CoV-2, which is particularly preferred, and/or the G614 variant of SARS-CoV-2, which is particularly preferred.

In another preferred of the method according to the present invention, said prevention and/or treatment is in combination with another antiviral therapy, for example selected from at least one remdesivir, interferon alpha 2a or 2b, inclusive any pegylated versions, chloroquine or hydroxychloroquine, serine protease inhibitors, cysteine protease inhibitors, and/or type II transmembrane protease (TMPRSS2) inhibitors, in particular camostat ((4-{2-[2-(Dimethylamino)-2-oxoethoxy]-2-oxoethyl}phenyl)(4-carbamimidamidobenzoate), inhalable corticosteroids, vasointestinal peptide (VIP) and furin inhibitors. Preferred is a combination with remdesivir, chloroquine or hydroxychloroquine, and/or camostat, which shows a synergistic effect.

In the method, the compound for use can be provided and/or is administered as a suitable pharmaceutical composition as discussed above. The compounds can be administered alone or in combination with other active compounds—for example with medicaments already known for the treatment of the aforementioned diseases, whereby in the latter case a favorable additive, amplifying or preferably synergistically effect is noticed. Suitable amounts and dosages to be administered to mammals, in particular humans, are as above.

In a preferred embodiment of the method according to the present invention, the compound is administered to said subject in an effective dosage, for example of between 0.1 to 10 mg/dose, preferably 0.2 to 5 mg per dose, which equals about 3 to about 80 μg per kilogram for a 70 kg subject per inhalation.

In the context of the method of the present invention, it was surprisingly found that the compound E64D shows particular advantages both in the prevention of viral infection as well as in the treatment of later stages of the diseases, particularly as a combination treatment. In the preventive approach of the method according to the present invention, the compound can be beneficially administered to a subject early in the context of the infection, as mentioned above. This regimen provides a surprisingly low viral load, and particularly prevents the progression of the infection to a more severe stage, such as the ones that may require mechanical ventilation.

In the treatment approach of the method according to the present invention, the compound can be administered to a subject in particular as a (synergistic) combination therapy, e.g. when administered at between 5 to 14 days post infection, preferably at between 5 to 11 days post infection, more preferably at 7 to 11 days post infection. This regimen provides a surprisingly effective treatment effect, and furthermore helps to control excessive immune reactions, like cytokine storms.

The present invention will now be described further in the following examples, nevertheless, without being limited thereto. For the purposes of the present invention, all references as cited herein are incorporated by reference in their entireties.

### EXAMPLES

### Cytotoxicity of E64D

E 64 D was found to be non-toxic in a cellular model (Vero e6-cells) in concentrations of at least 100 μM.

### Antiviral Effect of E64D-293 ACE2 Cells

The antiviral effect was measured by CytoPathicEffect (CPE) analysis of SARS-CoV-2 infected 293 ACE2 cells using crystal violet staining for surviving cells. The % of inhibition for the drug as tested was established in duplicate runs. The values were calculated from an OD of 0% inhibition (1% DMSO plus virus) to 100% (1% DMSO without any virus).

The antiviral activity was found to be at 77.9% and 67.9% inhibition without any substantial cytotoxicity (<4%). In view of this, it is anticipated to use even higher concentrations of more than 100 μM.

### Clinical Study on the Effectiveness of E64D in Human Patients

In this randomized, placebo-controlled trial safety and efficacy of E-64D is assessed in patients hospitalized for severe Covid-19. Patients are included in the study with confirmed SARS-CoV-2 infection who have an oxygen saturation of 94% or less while they were breathing ambient air or who were receiving oxygen support.

In more detail, patient inclusion parameters are:
Patients who had SARS-CoV-2 infection confirmed by reverse-transcriptase-polymerase-chain-reaction assay
oxygen saturation of 94% or less while the patient was breathing ambient air
a need for oxygen support
patients are required to have a creatinine clearance above 30 ml per minute and serum levels of alanine aminotransferase (ALT) and aspartate aminotransferase¹ less than five times the upper limit of the normal range
patients have to agree not to use other investigational agents for Covid-19

Clinical improvement is assessed in 50 patients receiving a 10-day course of inhaled E-64D three times a day, following 9 days. Follow-up will to continue through at least 28 days after the beginning of treatment with E64D or until discharge or death.

The primary end point is the time to clinical improvement, defined as the time from randomization to either an improvement of two points on a seven-category ordinal scale or discharge from the hospital. Secondary clinical end point is the reduction of viral load assessed by RT-PCR in the blood.

The solution formulation of E64D is supplied as a sterile, preservative-free, clear, colorless aqueous-based concentrated solution containing 50 μg/mL E64D. It is supplied as a sterile product in a single-use, clear glass vial with sufficient volume to allow withdrawal of 1.2 mL (60 μg E64D). In addition to the active ingredient, the solution formulation of E64D contains commonly used and pharmaceutically accepted ingredients for inhalation, such as a suitable buffer.

In the dosage regimen as applied, first, the above amount is inhaled in 6 healthy individuals (healthy volunteers) in order to exclude an adverse irritation of the airways. Lung function is assessed by bodyplethysmography before inhalation and after inhalation of E64D

Then, for the treatment of established CoV infection, including SARS-CoV, MERS-CoV, and SARS-2-CoV the dosage regimen is as follows: 3 times daily inhalation of 0.4 ml (15 to 30 minute period, total daily volume 1.2 ml) each time until the inhalation chamber is empty (the device has a sensor system that allows the device to shut down after complete inhalation). The recommended E64D dosing duration is a total of 10 days.

Inhaled doses are administered by an M-neb® dose device and over a 15 to 30 minute period three times a day. After inhalation, no residue remains in the nebulizer unit. The inhaler then turns off.

During treatment with E64D, the patient is admitted as an inpatient at a facility staffed and maintained by the requesting physician. A peripheral IV line or other venous catheter is maintained. Fluid resuscitation is available if necessary in the event of signs of renal failure or hypotension. Fever is treated with acetaminophen (up to maximum permitted daily dose) and antibiotics as indicated.

Use of nonsteroidal anti-inflammatory medications and other nephrotoxic agents is avoided, if possible.

The following laboratory tests are performed daily during therapy: serum chemistries including electrolytes, renal function tests (creatinine, CrCL, BUN), liver function tests (including ALT, AST, total bilirubin, and alkaline phosphatase), hematology (complete blood count and prothrombin time) and urinalysis.

CoV PCR is performed at regular intervals to monitor response to E64D therapy and to continually weigh the risks and benefits to the patient. Other lab and clinical parameters may be checked at the discretion of the physician.

Data on patients' oxygen-support requirements, adverse events, and laboratory values, including serum creatinine, ALT, and AST, are reported daily, from day 1 through day 10, and additional follow-up information is solicited through day 28.

For assessing the results, the incidence of the following key clinical events is quantified:
changes in oxygen-support requirements (ambient air, low-flow oxygen, nasal high-flow oxygen, noninvasive positive pressure ventilation [NIPPV], invasive mechanical ventilation, and extracorporeal membrane oxygenation [ECMO])
hospital discharge
reported adverse events, including those leading to discontinuation of treatment
serious adverse events
death

Furthermore, the proportion of patients with clinical improvement is evaluates, as defined by:
live discharge from the hospital
a decrease of at least 2 points from baseline on a modified ordinal scale (as recommended by the WHO R&D Blueprint Group). The six-point scale consists of the following categories: 1, not hospitalized; 2, hospitalized, not requiring supplemental oxygen; 3, hospitalized, requiring supplemental oxygen; 4, hospitalized, requiring nasal high-flow oxygen therapy, noninvasive mechanical ventilation, or both; 5, hospitalized, requiring invasive mechanical ventilation, ECMO, or both; and 6, death.
Virus copies in EDTA blood (before and after treatment)

E64D is permanently discontinued in the following conditions:
Development of ALT levels>5 times the upper limit of normal
Estimated creatine clearance<30 mL/min based on the Cockcroft-Gault formula

1. A pharmaceutical composition comprising a compound according to Formula (I), 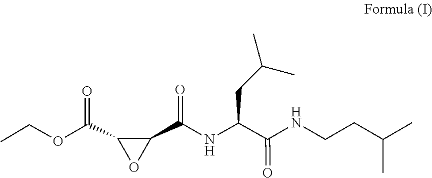 [SMILES: CCOC(=O)[C@H]1O[C@@H]1C(=O)N[C@@H](CC(C)C)C(=O)NCCC(C)C]
a physiologically acceptable salt, a solvate, or a hydrate thereof for use in the prevention and/or treatment of diseases caused by betacoronavirus infection in a mammalian subject, wherein said composition is formulated for administration by inhalation and comprises a suitable solvent as a carrier.

2-9. (canceled)

10. A method for preventing and/or treating a disease caused by betacoronavirus infection in a mammalian subject, comprising administering to said subject an effective amount of a compound according to Formula (I), 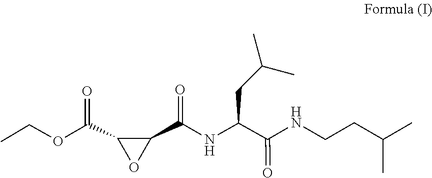 [SMILES: CCOC(=O)[C@H]1O[C@@H]1C(=O)N[C@@H](CC(C)C)C(=O)NCCC(C)C]
a physiologically acceptable salt, a solvate, or a hydrate thereof.

11. The method according to claim 10, wherein said method of preventing and/or treating is in combination with another antiviral therapy.

12. The method according to claim 10, wherein said compound is administered to said subject in an effective dosage of about between 1 and 100 mg/kg/day.

13. The method according to claim 10, wherein said compound is administered to said subject at between 5 to 14 days post infection.

14. The method according to claim 10, wherein for prevention said compound is administered to said subject at between 8 to 24 hours post infection.

15. The method according to claim 10, wherein said method further comprises a monitoring step comprising an analysis selected from serum chemistries, liver function tests, hematology, and urinalysis in a sample obtained from said subject, and comparing said analysis to the analysis of an earlier sample from said subject and/or a control sample.

16. The method according to claim 10, wherein said compound is administered by inhalation as a pharmaceutical composition comprising a suitable solvent as a carrier.

17. The method according to claim 16, wherein said suitable solvent is ethanol.

18. The method according to claim 10, wherein said disease is selected from respiratory disease, acute respiratory disease, sepsis, acute respiratory distress syndrome, and adverse immune reactions.

19. The method according to claim 10, wherein said infection is by HCoV-OC43, SARS-CoV-1, HCoV-HKU1, MERS-CoV, SARS-CoV-2 and/or the G614 variant of SARS-CoV-2.

20. The method according to claim 19, wherein said disease is caused by SARS-CoV-2.

21. The method according to claim 11, wherein said another antiviral therapy is selected from remdesivir; interferon alpha 2a or 2b; chloroquine or hydroxychloroquine; serine protease inhibitors; cysteine protease inhibitors; and/or type II transmembrane protease (TMPRSS2) inhibitors.
